(19) United States
(12) Patent Application Publication
(10) Pub. No.: US 2021/0187217 A1
(43) Pub. Date: Jun. 24, 2021

(54) PROCESS FOR OPERATING A MEDICAL DEVICE AND MEDICAL DEVICE OPERATING ACCORDING TO THE PROCESS

(71) Applicant: Drägerwerk AG & Co. KGaA, Lübeck (DE)

(72) Inventors: Birger LANDWEHR, Lübeck (DE); Michael GÖMANN, Hamfelde (DE)

(21) Appl. No.: 16/771,166

(22) PCT Filed: Dec. 12, 2018
(86) PCT No.: PCT/EP2018/084445

§ 371 Date: Jun. 9, 2020

(30) Foreign Application Priority Data
Dec. 18, 2017 (DE) 10 2017 011 684.9

(51) Int. Cl. A61M 16/00; G06F 3/01

(52) U.S. Cl. CPC A61M 16/0003; A61M 2016/1025; G06F 3/016; A61M 16/024

(57) ABSTRACT

A process for operating a medical device (10) includes applying a counter-torque, which acts during the rotation of a rotary knob (12) and depends on a current measured value (34). An actuator (20) applies the counter torque. A medical device (10) operates according to the process.

### CROSS REFERENCE TO RELATED APPLICATIONS

This application is a United States National Phase Application of International Application PCT/EP2018/084445, filed Dec. 12, 2018, and claims the benefit of priority under 35 U.S.C. § 119 of German Application 10 2017 011 684.9, filed Dec. 18, 2017, the entire contents of which are incorporated herein by reference.

### TECHNICAL FIELD

The present invention pertains to a process for operating a medical device and to a medical device operating according to the process.

### TECHNICAL BACKGROUND

A rotary knob with an actuating function is a known design feature of the Applicant's devices. The function is described, for example, in DE 195 00 529 C2. According to this, a graphic element (setter) is touched at first on a screen and the value of a parameter selected by means of the setter is then set by means of a rotary motion of the rotary knob to adjust parameters. The setting is concluded by pressing the rotary knob, when the set value for the particular parameter is taken over and becomes active for the device or system, i.e., for example, a ventilator. The adjustment of the value of a parameter takes place in a three-step procedure (“touch-turn-confirm”) by selecting the particular parameter by means of the setting, by a subsequent adjustment of the value of the parameter by means of a rotation of the rotary knob and by a final confirmation of the value set for the parameter by pressing the rotary knob.

These rotary knobs have, in general, no mechanical stop. The user can correspondingly rotate the rotary knob, in principle, endlessly in both directions (clockwise, counterclockwise). When the maximum value that can be set is reached, there is no change in the value any longer despite continued rotation. The user does not receive any mechanical feedback for this. He must rather observe a display of the set values during the adjustment. This also applies to so-called confirmation limits, by means of which the entire range that can be set is divided into a plurality of intervals. At the individual confirmation limits, the user must additionally actuate the rotary knob in order to be able to continue to adjust the value. For example, there are confirmation limits for the inspiratory ventilation pressure Pᵢₙₛₚ for 30 mbar, 50 mbar and 80 mbar.

A rotary knob is configured, in general, such that it provides a haptic feedback exclusively via mechanical locking for each incremental change of a parameter. During a slow rotation, the set value is changed usually by an individual increment, and by a plurality of increments in case of fast rotation.

Even though the operation of a medical device and the setting of a value of a parameter by means of a rotary knob is, on the whole, highly intuitive for the user, it is nevertheless still impossible to derive a more or less concrete set value from the position of the hand, which results during the actuation of the rotary knob, as this was possible, for example, in the past in the case of HiFi devices and a potentiometer acting as a volume control there with a corresponding rotary knob. Such a possibility of detecting a set value would, however, be important precisely when the user shall look at the patient during the adjustment of the value of a parameter (parameter adjustment). No haptic feedback has hitherto been available for the user when setting limits or actuating limits are reached, either.

In addition to the above-described three-step setting procedure, a selected parameter can also be changed within the framework of a so-called online adjustment directly and simultaneously with each incremental adjustment of the rotary knob. The set value consequently becomes active not only after a final confirmation, but already during the adjustment operation. This mode of setting is suitable, for example, in the case of so-called recruitment maneuvers, during which a change in pressure in the lungs accompanies each continuous incremental change of the particular setting value (for example, tidal volume). However, the user is forced due to the problem outlined above to look alternatingly at the medical device and the patient in order to be able both to reach the set value and to observe the effect of the set value on the patient.

Systems and applications with a forced feedback in connection with an operating action are established in many areas. They have been used for some years in the automobile industry, for example, in the form of a central rotary knob, for example, a rotary knob as is described in U.S. Pat. No. 6,686,911, of a user interface, in order to poll and perform different functions by hand. Systems with forced feedback (force feedback systems) are used in medical engineering where the user cannot have direct access to the location of the event, for example, during minimally invasive endoscopic surgery. Force feedback systems are also used to control surgical robots. The acting forces are fed back here for the user to the control element used, for example, a joystick.

### SUMMARY

One object of the present invention is to improve the possibility of operating a medical device, namely a medical device in which a rotary knob is provided for setting a value of a parameter, wherein the value of the parameter can be changed by changing an angle of rotation of the rotary knob.

This object is accomplished according to the present invention by means of an operating process having the features described in the independent process claim as well as by means of a medical device having the features of the independent device claim, which medical device operates according to the operating process and is therefore set up as intended.

Provisions are made in the process for operating a medical device for a counter-torque acting during the rotation of a rotary knob to be applied by means of an actuator and for the counter-torque to be dependent at least on a measured value, especially a measured value recorded in the medical device and/or a measured value recorded by means of the medical device. The measured value is, for example, a measured value that can be recorded by means of a sensor mechanism comprised by the medical device or associated with the medical device and is recorded during the operation of the medical device.

Due to the acting counter-torque, which counteracts a rotation of the rotary knob, being dependent at least on a measured value, the operator of the medical device receives an easy-to-interpret haptic feedback concerning the particular operating action performed by means of the rotary knob.

The advantage of the process being proposed is above all that the operator of the medical device can continue to look at the patient during the rotation of the rotary knob. It has hitherto been necessary for the operator of the medical device (clinician) precisely during setting operations during the treatment a patient and/or during therapeutic procedures at the patient to always keep looking away from the patient and to direct his view at the medical device instead. In addition to the drawback that the attention and concentration to the patient keep being interrupted hereby, the operator does not have, aside from the setting value being displayed, a directly usable haptic feedback, from which he could derive the approximate magnitude of the set value itself and the effect of this set value on the patient. Based on the counter-torque (“force feedback”), which is noticeable during the rotation of the rotary knob, the operator gets now, by contrast, a “feeling” for the set value.

Examples of medical devices that are in the foreground here are ventilators and anesthesia apparatuses.

A concrete example of a measured value, on which such a “perceptible” counter-torque depends, is an airway pressure, which is recorded by means of a pressure sensor, which is comprised by a medical device in the form of a ventilator or is associated with the medical device, namely, an airway pressure of a patient being ventilated by means of the ventilator. For example, a ventilation parameter, for example, a set point for an airway pressure, can be changed in a medical device in the form of a ventilator during the rotation of the rotary knob against the particular counter-torque. While rotating the rotary knob, the operator “feels” more or less the result of the particular setting being performed during an adjustment of this ventilation parameter and at a counter-torque, which depends on a measured airway pressure (actual value).

Based on this example, a preferred embodiment of the innovation being proposed should be highlighted, to which reference will often be made in the following description in the interest of better clarity, but without abandoning continued general validity. The following applies to this embodiment: The medical device is a ventilator or an anesthesia apparatus, hereinafter called summarily ventilator. The measured value, on which the counter-torque depends, is a measured value (actual value), which is recorded in relation to an airway pressure of a patient being ventilated by means of the ventilator. A ventilation parameter, for example, the tidal volume, which directly or indirectly determines or affects a set point of the airway pressure of the patient being ventilated by means of the ventilator, is set or adjusted during the rotation of the rotary knob.

The innovation being proposed here is expressly not limited to this example. The more general term measured value shall correspondingly always be implied whenever an actual value of an airway pressure (actual value of an airway pressure) is mentioned directly or generally. Likewise, the more general term of a particular set value of a ventilation parameter and in the same way the even more general term referring to the particular set angle of rotation of the rotary knob (which determines the set value of the ventilation parameter) shall always be implied whenever a set point of an airway pressure (airway pressure set point) is mentioned directly or generally. The description being presented here shall be interpreted in the sense that the terms and expressions implied are directly comprised by the description.

Features and details that are described in connection with said process for operating a medical device, especially for determining and setting a counter-torque noticeable during a rotation of a rotary knob, and in connection with possible embodiments, are, of course, also valid in the solution being proposed in connection with and in respect to a device intended for carrying out the process, namely, a medical device with means for carrying out the process and vice versa, so that reference is and can always mutually be made to the individual aspects of the present invention concerning the disclosure.

The process and embodiments of the process, which will be described below, and the process steps comprised thereby are carried out automatically, i.e., without intervention by a user of the particular medical device. The automatic performance of the process steps takes place under the control of a device control acting as a control unit of the medical device. This device control comprises, for example, a processing unit in the form of or in the manner of a microprocessor as well as a memory. A control program, which can be executed by the processing unit and which comprises an implementation of individual embodiments of the process or of a plurality of embodiments of the process and is executed during the operation of the medical device by the processing unit thereof, is or can be loaded into the memory.

The present invention is thus preferably implemented in software. The present invention is thus, on the one hand, also a computer program with program instructions executable by a computer and, on the other hand, a storage medium with such a computer program, i.e., a computer program product with program code means, as well as finally also a control unit or a medical device, into the memory of which control unit or medical device such a computer program is or can be loaded as a means for carrying out the process and embodiments thereof.

A counter-torque, which depends on the particular measured value and counteracts a rotation of the rotary knob, is applied by means of an actuator connected to the rotary knob in a non-positive manner in one embodiment of the process. An actuator, for example, an electric motor acting as an actuator, is a simple and easily controllable device for generating a counter-torque counteracting a rotation of the rotary knob.

In another embodiment of the process, the counter-torque (measured value-dependent counter-torque), which depends on a current measured value, is linked with a counter-torque (angle of rotation-dependent counter-torque), which depends on a current angle of rotation of the rotary knob. For example, end stops, confirmation limits or default values or suggested values can be signaled to the operator by means of a counter-torque dependent on the angle of rotation.

The above-mentioned object is also accomplished by means of a medical device, which is intended and set up for carrying out the process being here and hereinafter described and it thus has at least one rotary knob intended for setting a value of a parameter/ventilation parameter, wherein the value of the particular parameter/ventilation parameter can be changed by rotating the rotary knob, wherein at least one actuator is connected to the rotary knob in a non-positive manner, wherein a measured value can be detected by means of a sensor mechanism comprised by the medical device or associated with the medical device, and wherein a counter-torque, which depends on the detected measured value and counteracts a rotation of the rotary knob, can be applied by means of the actuator.

In one embodiment of the medical device, the actuator and an angle of rotation sensor are connected to the rotary knob in a non-positive manner. An angle of rotation of the rotary knob can be detected by means of the angle of rotation sensor. An additional counter-torque (angle of rotation-dependent counter-torque), which is superimposed to the counter-torque (measured value-dependent counter-torque) that depends on the measured value, can be determined on the basis of the angle of rotation. A counter-torque, which depends on the detected measured value and the detected angle of rotation and counteracts a rotation of the rotary knob, can then be applied by means of the actuator, and the user thus receives not only a haptic feedback for the particular measured value, i.e., for the result of the setting performed in the particular case, but, for example, also a feedback for angle of rotation-dependent confirmation limits or the like.

In another embodiment of the medical device, the angle of rotation sensor and the actuator are coupled with the rotary knob in a non-positive manner by means of a common shaft.

The non-positive coupling, on the one hand, of the angle of rotation sensor with the rotary knob and, on the other hand, of the actuator with the rotary knob is then brought about via one and the same component, namely, a common shaft.

An exemplary embodiment of the present invention will be explained in more detail below on the basis of the drawings. Mutually corresponding objects or elements are provided with the same reference numbers in all figures.

The exemplary embodiment shall not be considered to represent a limitation of the present invention. Rather, variations and modifications, especially such variants and combinations which the person skilled in the art can find in respect to accomplishing the object, for example, by a combination or variation of individual features contained in the general or special text of the description as well in the claims and/or in the drawings and lead to a new subject by combinable features, are possible within the framework of the present disclosure.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a device with a rotary knob;

FIG. 2 is a schematic view showing a medical device with an device according to FIG. 1;

FIG. 3 is a graph showing a counter-torque profile, which depends on a measured value and is noticeable during the rotation of the rotary knob;

FIG. 4 is a graph showing a counter-torque profile, which depends on a measured value and is noticeable during the rotation of the rotary knob;

FIG. 5 is a graph showing one of different angle of rotation-dependent counter-torque profiles;

FIG. 6 is a graph showing another of different angle of rotation-dependent counter-torque profiles;

FIG. 7 is a graph showing another of different angle of rotation-dependent counter-torque profiles;

FIG. 8 is a graph showing another of different angle of rotation-dependent counter-torque profiles;

FIG. 9 is a graph showing an angle of rotation-dependent counter-torque profile for signaling locking positions or default values or suggested values or value ranges;

FIG. 10 is a graph showing an angle of rotation-dependent counter-torque profile for signaling locking positions or default values or suggested values or value ranges;

FIG. 11 is a graph showing an angle of rotation-dependent counter-torque profile for signaling locking positions or default values or suggested values or value ranges; and

FIG. 12 is a graph showing an angle of rotation-dependent counter-torque profile for signaling locking positions or default values or suggested values or value ranges.

### DESCRIPTION OF PREFERRED EMBODIMENTS

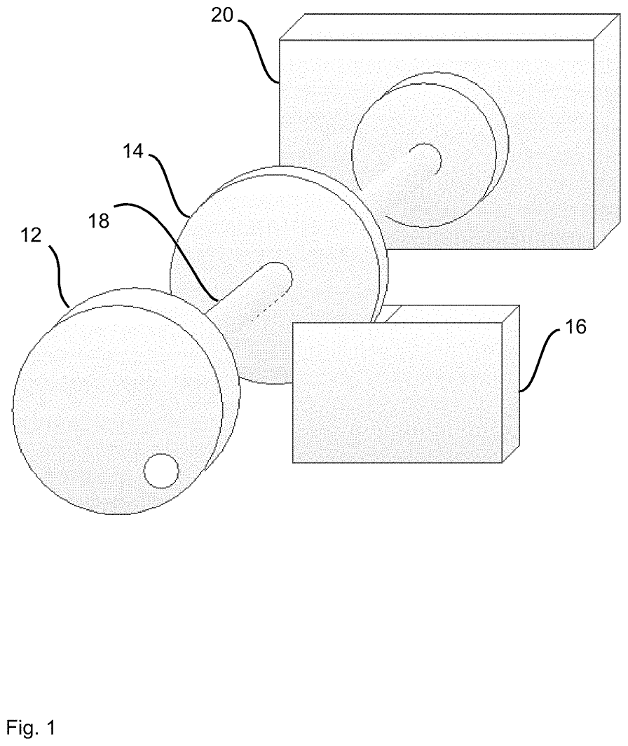

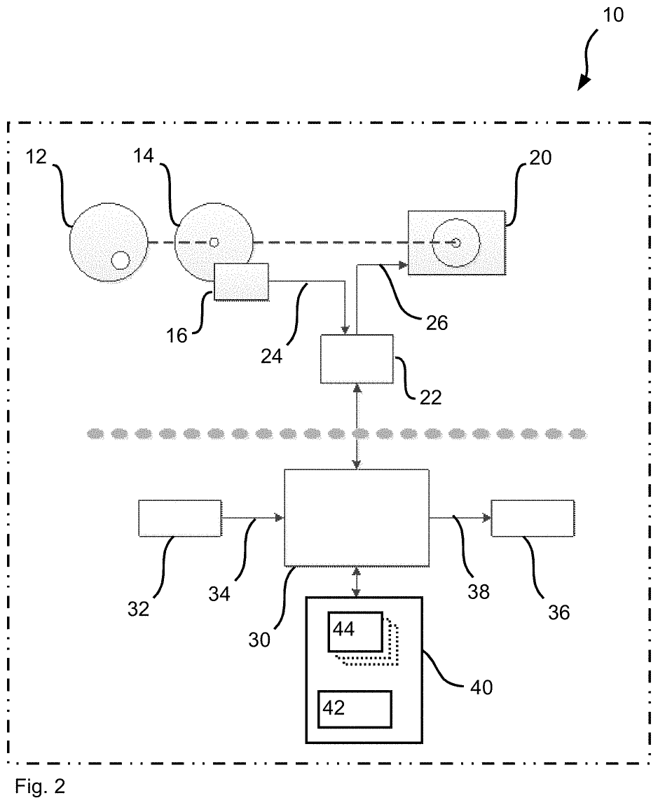

Referring to the drawings, the view in FIG. 1 shows in a schematically simplified form a device intended especially for a medical device 10 (FIG. 2), not shown here, for example, a ventilator, with a rotary knob 12 acting as an operating element of the medical device 10. An angle of rotation of the rotary knob 12, which will at times also be called position for short below corresponding to the general usage, is detected by means of a sensor mechanism. An angle of rotation sensor 16, which is associated with a disk 14 and which scans, for example, a material measure arranged on the disk 14 in a manner known, in principle, per se, is shown as an example of a sensor mechanism in the view in FIG. 1. The disk 14 is coupled (at least connected in a non-positive manner) to the rotary knob 12 via a shaft 18 or the like such that a rotation of the rotary knob 12 brings about a rotation of the disk 14, for example, by the disk 14 being arranged concentrically with the rotary knob 12 on a shaft 18 defining an axis of rotation of the rotary knob 12. As an alternative to a coupling by means of the axis of rotation acting as a common shaft 18, it is possible to use a frictional wheel or even a gear. An inductive sensor or a sensor with electrical sliding contact may also be considered for use as an angle of rotation sensor 16. Detection of the rotation and of a direction of rotation by means of a camera is possible as well. A value that can be detected by means of the angle of rotation sensor 16 concerning the position of the rotary knob 12 or of a plurality of rotations of the rotary knob 12 or of a speed of rotation of the rotary knob 12, etc., is considered to be the basis for the setting of a parameter of the medical device 10. However, such a derivation of a parameter that can be set by means of the rotary knob 12 is not in the foreground here and is, moreover, known per se. This will not correspondingly be considered here any further.

No additional opposed force is exerted so far during the adjustment of the rotary knob 12. The torque necessary for rotating the rotary knob 12 thus remains constant so far over the adjusted angle of rotation. The torque necessary for the rotation arises essentially from the frictional resistances of the mechanical configuration.

By contrast, provisions are made in the innovation being proposed here for the torque necessary for rotating the rotary knob 12 to vary depending on certain conditions. The operator of the medical device 10 is thus given a haptic feedback concerning the operating action performed during the rotation of the rotary knob 12.

A counter-torque acting as a force feedback is generated herefor by means of an actuator 20. The counter-torque must be overcome during the rotation of the rotary knob 12. Due to a value of the counter-torque, which depends, for example, on the position of the rotary knob 12, the operator receives a haptic feedback perceptible directly during the rotation of the rotary knob 12.

For example, an electromechanical drive, especially an electric motor, a braking device or a gel (magnetorheological liquid) with a viscosity that can be influenced by means of an electromagnetic field, acts as an actuator 20 for the force feedback. In the embodiment shown, the actuator 20 is coupled with the rotary knob 12 via the shaft 18. Provisions are made, in general, for the rotary knob 12 and the actuator 20 to be coupled directly or indirectly in a non-positive manner, for example, by means of a shaft 18, a frictional wheel, a gear or the like.

Reference is made to the view in FIG. 2 for the explanation of the function of the device according to FIG. 1. The rotary knob 12, the disk 14, the angle of rotation sensor 16 and the actuator 20 are shown in FIG. 2 as parts of the device according to FIG. 1 as components of a medical device 10 not shown in more detail, for example, of a ventilator. The at least non-positive coupling of the rotary knob 12 with the disk 14, on the one hand, and with the actuator 20, on the other hand, is shown in the view in FIG. 2 in the form of the broken line.

A control device 22 comprised by the medical device 10 receives sensor signals 24 from the angle of rotation sensor 16 and processes these. Within the framework of such a processing, the control device 22 continuously processes a respective direction of rotation of the rotary knob 12 and a change in the angle of rotation. To actuate the actuator 20, the control device 22 automatically generates control signals 26 for the actuator 20. This actuator 20 generates on the basis of a control signal 26 received a counter-torque, which is noticeable during the rotation of the rotary knob 12 based on the non-positive coupling between the actuator 20 and the rotary knob 12.

A device control 30 comprised by the medical device 10 is shown next to the control device 22 acting as an actuator control in the view shown in FIG. 2. The device control 30 acts as a central control unit of the particular medical device 10, i.e., for example, as a central control unit of a ventilator. The horizontal line between the area with the device control 30 and the area with the control device 22 shall illustrate a possible functional separation within the medical device 10. All components above this horizontal line are optionally combined in one assembly unit, in which the control device 22 thereof assumes an interface function to a higher-level unit, i.e., the device control 30 of the medical device 10 here. The device control 30 may also comprise, in principle, the control device 22 or at least the functionality of the control device 22 as well, so that the control device 22 does not appear as a separate functional unit. If the control device 22 is embodied (as is shown) within the medical device 10 independently from the device control 30, the device control 30 and the control device 22 are connected to one another in a communicating manner in the manner known per se, so that a data exchange from the device control 30 to the control device 22 and from the control device 22 to the device control 30 is possible.

The situation being shown (control device 22 independent from the device control 30 and connected to the device control 30 in a communicating manner) is assumed for the following description. A control device 22 comprised by the device control 30 or a device control 30 that comprises the functionality of the control device 22, for example, in software or in software and in firmware, shall always be implied and shall be considered with this reference as being comprised by the description being presented here.

A sensor mechanism 32, for example, a pressure sensor and/or a flow sensor or a plurality of pressure sensors and/or flow sensors, is associated at least functionally with the device control 30. The sensor mechanism 32 delivers during the operation at least one sensor signal, which codes a measured value 34. Furthermore, an actuator mechanism 36, for example, a valve acting as an actuator mechanism 36 or an actuator mechanism 36 comprising at least a valve, is associated with the device control 30 at least functionally. For actuating the actuator mechanism 6, the device control 30 generates, in a manner known, in principle, per se, at least one actuating signal 38, for example, actuating signals 38 that bring about an opening or closing of valves belonging to the actuator mechanism 36 in an inhalation branch and in an exhalation branch of a ventilator.

Different variants, of which two variants will be discussed below with further details and which will be called the first variant and the second variant for distinction, are considered for use for the automatic determination of a counter-torque belonging to the angle of rotation transmitted by the control device 22 by means of the device control 30.

In the first variant, which can also be considered to be a measured value-dependent variant and which leads to a measured value-dependent counter-torque, a counter-torque, which acts during the rotation of a rotary knob 12 and depends on a measured value 34 recorded by means of the sensor mechanism 32, is applied by means of the actuator 20.

In brief, the generation of a measured value-dependent counter-torque acting during the actuation of the rotary knob 12 by means of the actuator 20 comprises, for example, the following steps, which are carried out continuously or cyclically at regular, especially equally spaced times:
1. The device control 30 receives a sensor signal from the sensor mechanism 32.

The sensor signal codes a measured value 34.
2. The device control 30 automatically determines a torque (counter-torque) belonging to the measured value 34 obtained and transmits a datum coding the respective counter-torque to the control device 22.
3. The control device 22 generates a control signal 26 corresponding to the datum obtained and outputs this to the actuator 20.
The admission of the control signal 26 to the actuator 20 brings about the generation of the counter-torque by the actuator 20.

A control program 42, implemented in software and loaded into a memory 40, i.e., a computer program, which is executed during the operation of the medical device 10 by means of a processing unit comprised by the device control 30 in the form of or in the manner of a microprocessor, is used to carry out these steps in the embodiment shown. The memory 40 is either a memory 40 comprised by the device control 30 or a memory 40 accessible to the device control 30 in the usual manner.

The determination of a counter-torque belonging to the particular measured value 34 obtained (measured value-dependent counter-torque) by the device control 30 is carried out, for example, on the basis of a counter-torque profile 44 loaded into the memory 40. The counter-torque profile 44 may be formed by a parameter or a plurality of parameters in the simplest case. It can be determined by such parameters, for example, that the measured value 34 obtained is multiplied by a parameter of the counter-torque profile 44 and/or that a parameter of the counter-torque profile 44 is added to the measured value 34 obtained. The value range of the measured value 34 can be imaged by means of such or similar mathematical operations onto a value range of the counter-torque that can be exerted.

The function of the device control 30 can be described briefly such that the device control 30 “applies” the particular counter-torque profile 44 under the control of the control program 42, i.e., corresponding to the program code instructions comprised by the control program 42, when the control program 42 is executed during the operation of the medical device 10 by means of the processing unit comprised by the device control 30.

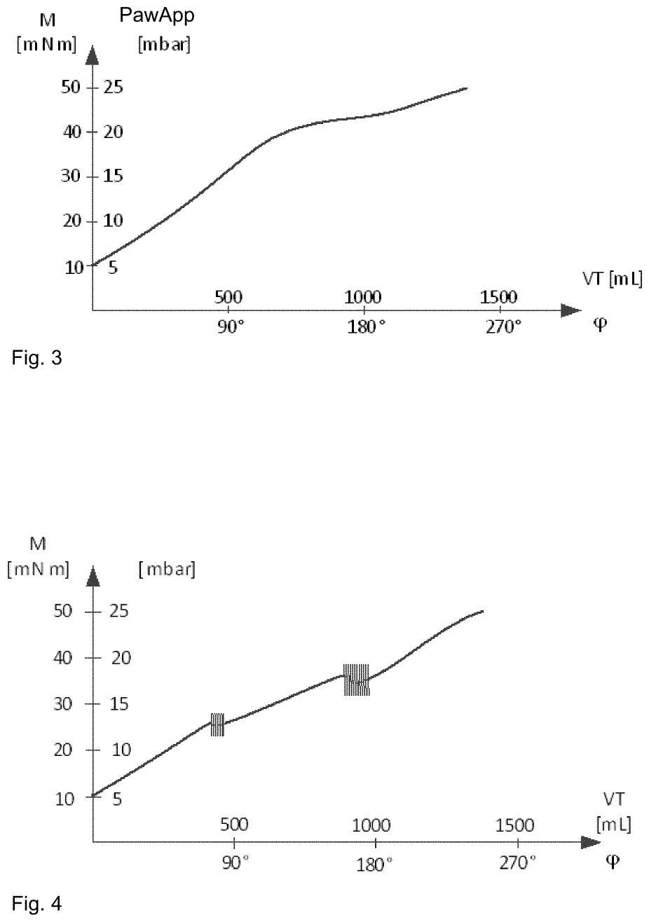

The view in FIG. 3 shows the relation between a ventilation parameter that can be set and a measured value 34 resulting for the ventilation parameter on the basis of a particular set value for a medical device 10 acting as a ventilator. In the view shown in FIG. 3, the ventilation parameter is the tidal volume VT and is plotted on the abscissa. The tidal volume VT is plotted in the unit milliliter [mL] and individual values (500 mL, 1,000 mL, 1,500 mL) are highlighted. The tidal volume VT set is obtained on the basis of a respective angle of rotation φ of the rotary knob 12. The measured value 34 resulting at the respective set tidal volume VT is an airway pressure PawApp recorded in the situation shown in FIG. 3 by means of a pressure sensor of the sensor mechanism 32, namely, an airway pressure PawApp of a patient being ventilated by means of the ventilator. The airway pressure PawApp is plotted on the ordinate in the unit millibar [mbar] and individual values (5 mbar, 10 mbar, 15 mbar, etc.) are highlighted. The ratio of the tidal volume VT to the airway pressure PawApp corresponds to the compliance of the patient's lungs. In the view in FIG. 3, the ratios are shown based on the example of the lungs of a healthy adult. The ratio of the tidal volume VT to the airway pressure PawApp is generally nonlinear.

In one embodiment of the approach being proposed here, the counter-torque M applied by means of the actuator 20 depends on the airway pressure PawApp, or generally on the particular recorded measured value 34. The counter-torque M is likewise plotted on the ordinate and is plotted in the units milliNewton-meter [mNm]. The counter-torque is obtained in the situation shown on the basis of a multiplication of the measured value 34, which has no unit, with a constant factor, namely, the factor 2. This factor is stored, for example, as a counter-torque profile 44, which is used automatically in the case of an adjustment of this ventilation parameter (tidal volume VT) and this measured value (airway pressure PawApp). A particular suitable counter-torque profile 44 may be provided for other conceivable pairings, and the automatic selection is preferably carried out under the control of the control program 42 by the device control 30 on the basis of the respective ventilation parameter, which can be varied by means of the rotary knob 12.

A mathematical function or a table may be used instead of a constant factor for determining the counter-torque M on the basis of the respective measured value 34, and, for example, a factor is obtained for imaging the value range of the measured value 34 onto the value range of the counter-torque or also directly the particular counter-torque M on the basis of the mathematical function or the table. Such a function or table or the like is likewise an example for a counter-torque profile 44.

On the basis of the counter-torque M, which depends on the particular measured value 34, the user receives a haptic feedback concerning the result of the value set for the particular ventilation parameter during the rotation of the rotary knob 12.

The advantage of such a haptic feedback can be illustrated especially clearly on the example of a so-called recruitment maneuver. During such a recruitment maneuver, the operator can quasi “sense” compliance changes, which arise from the opening of collapsed lung regions (atelectases), during the rotation of the rotary knob 12. Two areas with greatly fluctuating airway pressure PawApp values resulting from such compliance changes are shown in the view shown in FIG. 4. A proportional oscillation of the counter-torque is associated with the fluctuating (oscillating) airway pressure PawApp, which the operator perceives in a narrowly limited angle of rotation range during the rotation of the rotary knob 12. In case of a first, abrupt increase in the counter-torque in such an area, the operator may cautiously feel around to some extent by continuing to turn the rotary knob 12. The opening of a lung region as a result of the continued turning of the rotary knob 12 can be detected from an abrupt reduction of the counter-torque. When this phenomenon (perceptible increase and abrupt drop in the counter-torque) recurs, the operator recognizes the opening of additional lung regions. If, by contrast, there is no drop in the counter-torque and a further increase in the counter-torque is rather observed, the operator recognizes that it was not possible to achieve any opening of atelectases within the framework of the recruitment maneuver or no opening of atelectases could apparently be achieved and other measures shall then be taken.

The trigger of spontaneous breathing may optionally be given in the form of a pulse applied to the rotary knob 12 at the time of each online adjustment of a ventilation parameter. An indication of spontaneous breathing of the patient can be derived for this from a corresponding measured value 34. A brief (pulse-like) increase in the counter-torque may be superimposed, for example, to a counter-torque according to FIG. 3.

In the above-mentioned second variant, which may also be considered to be an angle of rotation-dependent variant and leads to a rotary knob-dependent counter-torque, a counter-torque, which acts during the rotation of a rotary knob 12 and depends on a current value based directly on a setting made by an operator, is applied during the rotation of a rotary knob 12.

The measured value-dependent counter-torque depends indirectly rather than directly on the setting made by the operator, since the underlying measured value 34 is recorded, for example, at the patient.

The current value, on which the counter-torque depends in this second variant, is an angle of rotation of the rotary knob 12, a set value resulting from the angle of rotation of the rotary knob 12 or a value range of the angles of rotation or a value range of the resulting set values. In case the counter-torque depends on a value range of the angles of rotation, the counter-torque likewise depends on the respective angle of rotation, since the particular angle of rotation determines whether or not this belongs to a certain value range. In case of a dependence of the counter-torque on a set value resulting from the angle of rotation, the counter-torque depends at least indirectly on the angle of rotation and hence likewise on the angle of rotation, since the angle of rotation determines the set value. In case of a dependence of the counter-torque on a value range of the set values, the counter-torque likewise depends at least indirectly on the angle of rotation, since the angle of rotation determines the set value and it thus determines whether or not this set value belongs to a certain value range. An angle of rotation-dependent counter-torque will at times be referred to briefly in the following description in the interest of better clarity and the description will be continued on this basis. All other possibilities described shall always be implied and shall be considered with this reference as being comprised by the description being presented here.

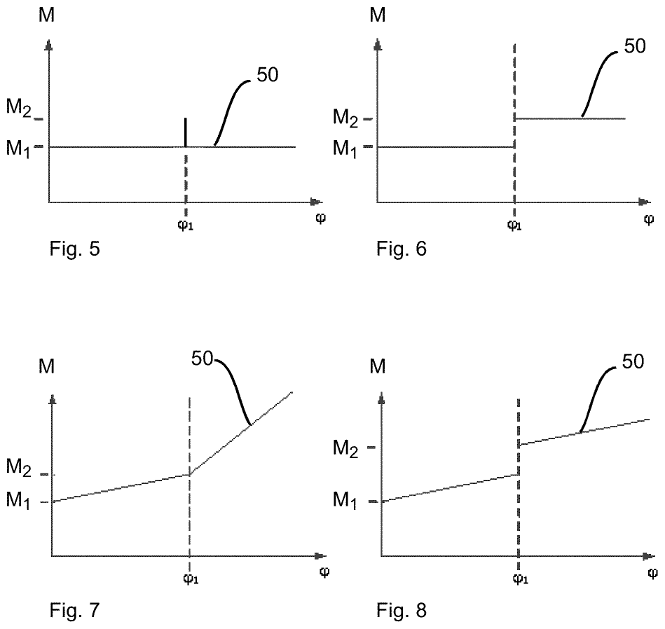

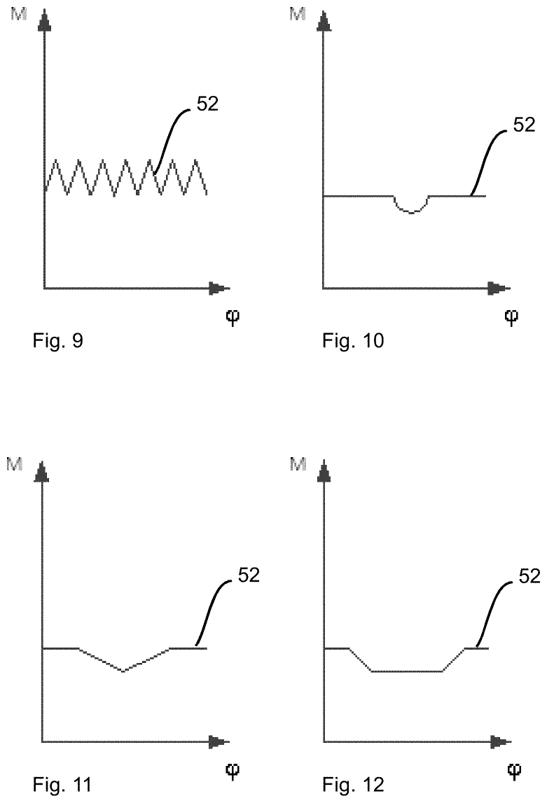

An angle of rotation-dependent counter-torque may optionally be superimposed to a measured value-dependent counter-torque, and the views shown in FIG. 5 through FIG. 8 as well as in FIG. 9 through FIG. 12 show examples of angle of rotation-dependent counter-torques. A counter-torque profile 44 underlying such an angle of rotation-dependent counter-torque can also be stored in the memory 40 and the counter-torque profile 44 is “applied” by the device control 30, as outlined above, and the device control 30 superimposes the measured value-dependent counter-torque profile 44 and the angle of rotation-dependent counter-torque profile 44 or the respective resulting counter-torques.

The operator of the medical device 10 can be given, for example, a haptic feedback in relation to confirmation in addition to the haptic feedback of the effect resulting from the setting performed during the rotation of the rotary knob 12 by means of a superimposition of a measured value-dependent counter-torque to an angle of rotation-dependent counter-torque.

Confirmation limits serve basically the purpose of reliably setting parameter values. It is avoided with a confirmation limit, for example, that an excessively high set value is taken over for the therapy in case of an excessively fast rotation and of a possibly unintended confirmation. Precisely when the rotary knob 12 has the function of a pressure-setting unit, this could possibly lead to a serious risk for the patient. The entire setting range is therefore divided into individual intervals. A changeover from one interval into the next interval is currently possible only after an additional confirmation. However, the fact that a confirmation limit is reached can currently be recognized only visually from the fact that a set value will not change any more even when the rotary knob 12 continues to be rotated and a corresponding additional message will possibly appear. To recognize the fact that a confirmation limit has been reached, the user must consequently look at the device rather than at the patient during the setting operation.

According to the approach being proposed here, the fact that a confirmation limit has been reached and possibly exceeded is optionally indicated to the user by means of a noticeable additional counter-torque (in addition to the counter-torque resulting from the respective measured value 34). Corresponding counter-torque profiles 44, some of which are shown as examples in FIGS. 5 through 7 to illustrate the intended basic principle, are provided as the basis for such a counter-torque.

The view in FIG. 5 shows as a counter-torque profile 44 a graph 50 with a pulse-like increase at a certain angle of rotation (φ₁). Until this angle of rotation is reached, there is a (preset or presettable) first counter-torque M₁ during the rotation of the rotary knob 12. A (preset or presettable) higher, second counter-torque M₂ acts when this angle of rotation is reached. The initial, first counter-torque M₁ will again be present after exceeding this angle of rotation. The pulse-like increase in the counter-torque makes it possible to notice the fact that the confirmation limit has been reached and exceeded during the rotation of the rotary knob 12. For the sake of brevity, it can also be said that the pulse-like increase in the counter-torque forms the confirmation limit.

The following description will be continued on the basis of the fact that a noticeable change in the counter-torque, occurring at a certain angle of rotation, and/or following a certain angle of rotation, indicates a confirmation limit and thus acts as a confirmation limit in the sense of a limit that is to be overcome. Furthermore, it should be pointed out, insofar as referring to a counter-torque acting on the basis of the respective counter-torque profile 44 in connection with the explanation of FIGS. 5 through 8 as well as FIGS. 9 through 12, that this is meant to be exclusively a counter-torque acting on the basis of the respective (angle of rotation-dependent) counter-torque profile 44. The value of the actually acting counter-torque is usually different from this based on the superimposition of a measured value-dependent counter-torque or counter-torque profile 44 to at least one angle of rotation-dependent counter-torque or counter-torque profile 44.

Based on the fact that the second counter-torque M₂ is higher in the counter-torque profile 44 according to FIG. 5, it is necessary to apply a stronger force to overcome this confirmation limit during the continued rotation of the rotary knob 12 at the angle of rotation φ₁ at the confirmation limit thus obtained. Unintentionally overcoming the confirmation limit and unintentionally entering the range following the confirmation limit can thus be effectively avoided because the operator will always notice the reaching of the confirmation limit and the possible exceeding of the confirmation limit during the rotation of the rotary knob 12 based on the change in the effective counter-torque.

Instead of the situation shown in FIG. 5 as an example with exactly one confirmation limit, a plurality of confirmation limits located at regularly or irregularly spaced locations over the entire setting range, which can be selected by means of the rotary knob 12, are possible as well. This also applies to all the examples described below.

The view in FIG. 6 shows the graph 50 of another variant of a counter-torque profile 44 intended to embody a confirmation limit at a certain angle of rotation (φ₁). In front of and behind the confirmation limit, the respective effective counter-torque (M₁ or M₂) is constant, but the level is markedly higher (M₂>M₁) behind the confirmation limit. While rotating the rotary knob 12, the operator notices that the confirmation limit is reached on the basis of the abruptly higher counter-torque (M₁ before; M₂ now). In addition, a continued rotation of the rotary knob 12 in the area following the confirmation limit is markedly more difficult because of the higher counter-torque (M₂) acting there than in the area in front of the confirmation limit.

In the graph 50 shown in FIG. 7 and in the counter-torque profile 44 based on this, the confirmation limit is obtained by the counter-torque M increasing from an initial counter-torque M₁ to a counter-torque M₂ (M₂>M₁) at the confirmation limit as a function of the angle of rotation and at a first pitch m₁. Following the confirmation limit, the counter-torque M increases at a higher pitch m₂ (₂>m₁) beginning from the counter-torque M₂ acting at the confirmation limit. During the rotation of the rotary knob 12 in the direction of the confirmation limit, the operator notices the progression in the area in front of the confirmation limit based on the continuously increasing counter-torque (pitch m₁). The exceeding of the confirmation limit can be noticed directly from the fact that a continued rotation of the rotary knob 12 following the confirmation limit is markedly more difficult because of the higher pitch m₂ acting there (m₂>m₁) and the more rapid increase in the counter-torque resulting therefrom.

In the situation shown in FIG. 8, the confirmation limit is embodied more or less in the form of a combination of the conditions in FIG. 6 and the angle of rotation-dependent increase in the counter-torque according to FIG. 7. The counter-torque M increases up to a certain angle of rotation φ₁ starting from an initial counter-torque M₁ in an angle of rotation-dependent manner and with a first pitch m₁. The counter-torque increases abruptly at the confirmation limit to a higher counter-torque M₂ (M₂>M₁). Following the confirmation limit, the counter-torque M increases further from the counter-torque M₂ acting at the confirmation limit with a pitch m₁ acting in front of the confirmation limit. While rotating the rotary knob 12, the operator notices the progression in the area in front of the confirmation limit based on the continuously increasing counter-torque. The operator notices the fact that the confirmation limit has been reached from the abruptly higher counter-torque. Further rotation of the rotary knob 12 is markedly more difficult following the confirmation limit because the counter-torque is markedly higher there even initially than in the area in front of the confirmation limit, and, in addition, the counter-torque continues to increase in the area following the confirmation limit, optionally, for example, unlike as shown, with a higher pitch than in front of the confirmation limit.

In one mode of operation, in which two or more parameter values are adjusted simultaneously and synchronously, this can optionally be signaled to the operator by an increased counter-torque. In such a mode of operation and in such a coupling, the operator rotates two or more rotary knobs quasi simultaneously. This coupling and the resulting “simultaneous rotation of a plurality of rotary knobs” can be signaled to the operator by a correspondingly higher counter-torque, which is noticeable during the rotation. The coupled parameters, for example, respiration rate and inhalation time, are selected for such a coupling, for example, by actuating corresponding setters of an operating surface of the medical device 10.

For example, a superimposition of two measured value-dependent counter-torque profiles 44 or a superimposition of a measured value-dependent counter-torque profile 44 to one of the angle of rotation-dependent counter-torque profiles 44 shown in the view in FIG. 6 through FIG. 8 may be employed in such a coupling. The switching to a higher counter-torque (FIG. 6, FIG. 8) and/or a switching to a respective pitch of the counter-torque (FIG. 7, FIG. 8) does not depend now, unlike in the views shown in FIGS. 6 through FIG. 8, on an angle of rotation preset or presettable as a confirmation limit. The switching rather depends on the beginning of the coupling, i.e., on a corresponding time t. The angle of rotation φ(t) of the rotary knob 12 being actuated, which angle is given at the time t, is the angle of rotation φ₁ plotted in the views shown in FIG. 6 through FIG. 8 in such a situation. The switching (increasing the counter-torque and/or increasing the pitch) may optionally be dependent on the number of coupled parameters, such that the force necessary to overcome the effective counter-torque during the rotation of the actuated rotary knob 12 increases as a function of the number of the parameters coupled.

The views in FIG. 9 through FIG. 12 show the graphs 52 of other, basically optional angle of rotation-dependent counter-torque profiles 44, which may optionally be combined with a measured value-dependent counter-torque profile 44, but also with a measured value-dependent counter-torque profile 44 and with the angle of rotation-dependent counter-torque profiles 44 shown and described hitherto (FIG. 5 through FIG. 8). A counter-torque profile 44 based on the graph 52 shown in FIG. 9 imitates a hitherto mechanically implemented locking function of the rotary knob 12. The hitherto necessary corresponding mechanical element can thus be eliminated. Instead of the triangular function shown, a counter-torque profile 44 based on a so-called sawtooth may, for example, also be considered for use as an alternative. The triangles or saw teeth in the counter-torque profile 44 also do not have to absolutely follow one another directly. An area with a constant counter-torque may rather be present between such changes of the effective counter-torque, which signal the locking of the rotary knob 12 at a certain angle of rotation.

The graphs 52 in FIG. 10, FIG. 11 and FIG. 12 show examples for counter-torque profiles 44 for signaling default values or suggested values. In order to signal to the operator of the medical device 10, for example, a recommended set value or setting range, this may be carried out by means of a counter-torque profile 44 with a simulated trough (FIG. 11), a depression (FIG. 11) or tub (FIG. 12), i.e., the adjusting force becomes minimal during the corresponding setting based on the locally reduced counter-torque. Starting from this point, the force necessary for overcoming the effective counter-torque increases in both directions.

The graphs 50, 52 shown in the views in FIG. 5 through FIG. 8 as well as in FIG. 9 through FIG. 12 are likewise examples of angle of rotation-dependent counter-torque profiles 44, which are based on the graphs 50, 52 and can be applied by the device control 30, for converting an angle of rotation of the rotary knob 12 into a corresponding counter-torque applied by means of the actuator 20. The respective counter-torque profiles 44 may be stored in the memory 40 in the form of a mathematical function, of a plurality of mathematical functions, a table or the like or in the form of the particular parameters to be determined (counter-torque values M₁, M₂ and/or pitches m₁, m₂; distances between two counter-torque values M₁, M₂ and/or pitches m₁, m₂; ratios of two counter-torque values M₁, M₂ and/or pitches m₁, m₂).

The basic possibility of combining the counter-torque profiles 44 shown and explained should finally be pointed out once again. For example, a measured value-dependent counter-torque profile 44 according to FIG. 3 or FIG. 4 can be complemented by a locking function according to FIG. 9 and/or by a signaling of default values or suggested values or value ranges according to FIG. 10 through FIG. 12.

The reaching of a minimally or maximally settable value (end stop) of a ventilation parameter may optionally also be signaled by means of a counter-torque that becomes active when the end stop is reached. The counter-torque signaling an end stop is preferably a maximally applicable counter-torque. A corresponding angle of rotation-dependent counter-torque profile 44 is used for this, which is optionally superimposed with other angle of rotation-dependent counter-torque profiles 44 to a measured value-dependent counter-torque profile 44. The counter-torque profile 44 bringing about a one-side end stop or a two-side end stop leads to an especially high preset or presettable counter-torque, especially the maximum applicable counter-torque, at an angle of rotation of the rotary knob 12, which angle of rotation corresponds to or goes beyond the respective end stop.

For the angle of rotation-dependent counter-torque, reference is made concerning further details to the simultaneous application of the same applicant to International Application publication WO2019121185A1, which shall be considered with this indication to be included with its full disclosure content in the description being submitted here in order to avoid further repetitions.

Individual aspects of the description being submitted here, which are in the foreground, and of the measured value-dependent counter-torque, which is in the foreground, can thus be briefly summarized as follows: Proposed are a process for operating a medical device 10, wherein counter-torque, which acts during the rotation of a rotary knob 12 and depends on a current measured value 34, is applied according to the process by means of an actuator 20, as well a medical device 10, which operates according to the process and is thus set up as intended.

The measured value 34, for example, a measured value for a ventilation pressure, is an indicator of an effect, which becomes established on the basis of a setting of a ventilation parameter in the patient. The counter-torque, which is applied by means of the actuator 20 and acts during the rotation of the rotary knob 12, depends directly or indirectly on the respective set value of the ventilation parameter and imparts to the operator a feeling for the effect associated with an operating action.

While specific embodiments of the invention have been shown and described in detail to illustrate the application of the principles of the invention, it will be understood that the invention may be embodied otherwise without departing from such principles.

1. A process for operating a medical device, the process comprising the steps of:
providing the medical device with a rotatable rotary knob;
providing an actuator for applying a torque to the rotary knob;
providing a sensor mechanism comprised by the medical device or associated with the medical device;
applying a counter-torque to the rotary knob with the actuator during a rotation of the rotary knob by a user applying a torque to the rotary knob, wherein the counter-torque depends on a measured value recorded by the sensor mechanism.

2. A process in accordance with claim 1, wherein the measured value is an actual value of a ventilation parameter.

3. A process in accordance with claim 2, wherein a set point of a ventilation parameter, which set point determines or influences the actual value of the ventilation parameter, is set with the rotation of the rotary knob.

4. A process in accordance with claim 1, wherein the counter-torque, which depends on the detected measured value and counteracts a rotation of the rotary knob, is applied by the actuator and the actuator is non-positively connected to the rotary knob.

5. A process in accordance with claim 1, wherein:
the measured value recorded by the sensor mechanism is a current measured value;
the counter-torque depending on the current measured value is linked with a counter-torque dependent on a current angle of rotation of the rotary knob;
the counter-torque dependent on the current angle of rotation of the rotary knob is determined on the basis of at least one counter-torque profile.

6. A process in accordance with claim 5, wherein a plurality of counter-torque profiles are combined to determine a counter-torque dependent on a current angle of rotation of the rotary knob.

7. A medical device device comprising:
at least one rotary knob for setting a value of a parameter, wherein the value of the parameter is varied by rotating the rotary knob;
an actuator non-positively connected to the rotary knob; and
a sensor mechanism for detecting a measured value, the sensor mechanism being comprised by the medical device or associated with the medical device, and wherein a counter-torque, which depends on the detected measured value and counteracts a rotation of the rotary knob, is applied by the actuator.

8. A medical device in accordance with claim 7, further comprising an angle of rotation sensor, wherein:
the actuator and the angle of rotation sensor are non-positively connected to the rotary knob;
an angle of rotation of the rotary knob is detectable by the angle of rotation sensor; and
the counter-torque, which depends on the detected measured value and on the detected angle of rotation and counteracts a rotation of the rotary knob, is applied by the actuator.

9. A medical device in accordance with claim 8, wherein the angle of rotation sensor and the actuator are non-positively coupled with the rotary knob by means a common shaft.

10. A process according to claim 1, wherein a computer program with program code means controls the application of the counter-torque to the rotary knob with the actuator acting during the rotation of the rotary knob by the user applying a torque to the rotary knob when the control program is executed by a device control controlling and/or monitoring the medical device.

11. A medical in accordance with claim 7, further comprising a device control configured to control and/or monitor the medical device including to control the application of the counter-torque to the rotary knob with the actuator acting during the rotation of the rotary knob by the user applying a torque to the rotary knob.

12. A medical device in accordance with claim 11 wherein the device control comprises a memory or the memory is associated with the device control and a control program can be loaded into the memory.

13. A medical device in accordance with claim 11, wherein medical device comprises a ventilator and the sensor mechanism is configured to measure an actual value of a ventilation parameter as the measured value.

14. A medical device in accordance with claim 13, wherein a set point of a ventilation parameter, which set point determines or influences the actual value of the ventilation parameter, is set with the rotation of the rotary knob.

15. A medical device in accordance with claim 11, wherein the actuator is non-positively connected to the rotary knob.

16. A medical device in accordance with claim 11, wherein:
the measured value recorded by the sensor mechanism is a current measured value;
the counter-torque depending on the current measured value is linked with a counter-torque dependent on a current angle of rotation of the rotary knob;
the counter-torque dependent on the current angle of rotation of the rotary knob is determined on the basis of at least one counter-torque profile.

17. A medical device in accordance with claim 16, wherein a plurality of counter-torque profiles are combined to determine a counter-torque dependent on a current angle of rotation of the rotary knob.
